(19) United States
(12) Patent Application Publication
(10) Pub. No.: US 2010/0215725 A1
(43) Pub. Date: Aug. 26, 2010

(54) PHARMACEUTICAL COMPOSITION CONTAINING IDEBENONE FOR THE TREATMENT OF LIVER DISORDERS

(76) Inventors: Joseph Schwarz, Markham (CA); Michael Weisspapir, Markham (CA)

Correspondence Address: ALPHARX INC., 168 KONRAD CRESCENT, SUITE 200, MARKHAM, L3R 9T9, CA

(21) Appl. No.: 12/389,684

(22) Filed: Feb. 20, 2009

(51) Int. Cl. A61K 31/122; A61K 9/127; A61K 9/50; A61K 9/14; A61P 1/00

(52) U.S. Cl. 424/450; 514/688; 424/502; 424/490

(57) ABSTRACT

The invention describes the use of an injectable form of Idebenone to protect against hepatic damage, improve recovery from liver trauma, poisoning, vapor intoxication, degenerative diseases, hepatocyte function loss and pathology associated with inflammation or infection. The use of injectable Idebenone restores liver function, suppresses elevated enzyme levels, decreases alcoholic and drug abuse associated syndromes, symptoms of acute hepatitis of various origins, the consequences of liver reperfusion and other signs of liver damage.

### FIELD OF INVENTION

The invention relates to the field of preparation of stable formulations of Idebenone suitable for parenteral administration. Existing oral dosage forms of Idebenone are associated with high metabolization in the liver (“first pass effect”) and cannot be administered in acute situations or in cases of patient unconsciousness. The development of an injectable form of Idebenone is highly desirable.

### BACKGROUND OF THE INVENTION

Liver damage associated with various diseases, syndromes and many other conditions (acute liver failure, acute hepatitis, elevated hepatic enzymes, liver injury and trauma, liver infarction, cirrhosis, paracetamol poisoning, alcoholic intoxication, post-anesthesia hepatic damage) are in need of effective treatment and prophylaxis [1].

Chemicals often cause sub-acute liver injury manifested as abnormal liver enzyme tests, but with no discernable clinical symptoms. More than 900 drugs have been implicated in causing liver injury. Drug induced liver injury is responsible for 5% of all hospital admissions and 50% of all acute liver failures [2].

Liver damage is also closely correlated with alcohol abuse, viral hepatitis (especially A and B types), liver transplantation, the use of such drugs as antibiotics (tetracyclines), tuberculocidal agents (isoniazide), NSAID analgesics (paracetamol, acetaminophen, salicylates and metamizole), organic solvents (chloroform, carbon tetrachloride, dichloroethane, toluene, etc.), inhalational anesthetics (isoflurane, enflurane), aflatoxins and many other hepatotoxic substances, as well as cholestatic conditions, liver reperfusion or acute inflammation associated with viral infection.

There is serious need for effectively treating these conditions, and yet adequate treatment options do not currently exist.

Apoptotic hepatocyte cell death is the fundamental cause of acute and chronic liver diseases [3]. In hepatocytes, TNF- or Fas receptor-mediated apoptotic cell death is dependent on mitochondria, to amplify the initial receptor-derived death signal [4, 5].

In many cases, liver damage is associated with a lack of oxygen caused by decreased blood circulation and accompanied by an excess of free radicals, which suppress mitochondrial function.

Antioxidants have shown promise as protective agents against diminished brain function, resulting from extended general anesthesia associated with major surgical procedures in the elderly. Various substances—antioxidants and radical scavengers—have been tested in in vitro cell cultures, ex vivo brain slices and in vivo animal models. In such experiments, Idebenone, 2,3-dimethoxy-5-methyl-6-(10-hydroxydecyl)-1,4-benzoquinone, demonstrated pronounced antioxidant activity and marked protection against oxidative damage to brain cells. An oral form of Idebenone is used for treatment of cardiac muscle atrophy in Friedreich's Ataxia [U.S. Pat. No. 6,133,322 by Rustin P., et al. “Quinone derivatives for treating or preventing diseases associated with iron overload.”] as a cell protectant and to some extent, in the treatment of Alzheimer's Disease [U.S. Pat. No. 5,916,925, by Higuchi S., “Pharmaceutical composition for treatment of dementia”].

In a study of nine patients with cerebrovascular disease, 90 mg of Idebenone was given daily and electroencephalograms and clinical symptoms were monitored. The results suggested that Idebenone supplementation produced improvements in EEG and clinical symptoms in these patients [3].

Idebenone protects cultured cortical neurons against necrotic degeneration; it rescues cortical neurons even when applied 30 min after the NMDA pulse, suggesting that the drug interferes with the chain of toxic reactions triggered by an excessive stimulation of excitatory amino acid receptors [4].

Idebenone oral dosing (5 mg/kg daily for 8 weeks) in Friedreich's Ataxia patients significantly decreases a marker of oxidative DNA damage. Idebenone has been shown to prevent iron-induced lipoperoxidation and cardiac muscle injury in three patients given 5 mg/kg daily for 4-9 months, resulting in a reduction of left ventricular enlargement in these individuals [6].

In cell culture experiments, Idebenone has been shown to scavenge a variety of free radical species [7]. It can also redox couple with hypervalent species of myolglobin and hemoglobin, thus preventing lipid peroxidation promoted by these species. Likewise, Idebenone inhibits microsomal lipid peroxidation induced by ADP-iron complexes or organic hydroperoxides. In so doing, it prevents the destruction of cytochrome P450, which would otherwise accompany lipid peroxidation.

The ability of Idebenone to protect against liver lipid peroxidation and protein damage mediated by the pro-oxidative system NADPH/ADP/Fe3+ was tested in a rat liver microsomal model [8]. Idebenone, in concentrations of 20 micromol/L vs coenzyme Q-10 100 micromol/L, offered complete protection against lipid peroxidation in microsomes. The use of Idebenone during liver transplantation may increase donor organ preservation by maintaining organ quality and preventing reperfusion injury.

A permanently charged triphenylphosphonium derivative of coenzyme Q10, mitoquinone (MitoQ), has been proposed as a treatment for liver damage associated with hepatitis C viral infection and alcoholic steathitis [U.S. Pat. No. 7,232,809 by Murphy M. “MITOCHONDRIALLY TARGETEED ANTIOXIDANTS.”]. In clinical trials, mitroquinone was administrated orally in capsules of 40 and 80 mg [9].

The bioavailability of oral Idebenone is relatively high, due to its polar hydrophobic nature. However, the oral administration of Idebenone is accompanied by a pronounced first pass metabolism in the liver and small quantities of drug are subsequently available to the brain or other targeted organs. Additionally, the effects of an oral treatment regimen only become apparent after weeks or even months of drug administration.

An injectable form of Idebenone overcomes the first pass effect of an oral dosage form and rapidly provides the required concentration in blood and brain tissue. Nevertheless, no parenteral Idebenone dosage form currently exists. The only successfully documented intravenous administration of Idebenone involves an experiment in rats, utilizing a 10% solution of polyethoxylated castor oil surfactant [10], which can not be applied to human use due to the hemolytic properties of the carrier vehicle.

The low water solubility of Idebenone makes this task very difficult. The use of water miscible solvents (alcohol, propylene glycol, liquid PEG, N-methylpyrrolidone, etc.) in which the drug dissolves well are inappropriate for injection, due to its immediate precipitation upon contact with physiological fluids or a water phase. An inclusion complex of Idebenone with cyclodextrin has been described, but it is water dispersible, not soluble, and therefore, not suitable for injection. The solubility of Idebenone in fixed oils (soy, corn, almond, etc.) is low. The drug precipitates from such emulsions during storage, limiting their use for an injectable formulation. A combination of solvents, oils and surfactants results in emulsions with a relatively large droplet size in vivo, making them unsuitable for intravenous delivery.

### DESCRIPTION OF THE INVENTION

Fas/APO-1 (CD95), an apoptosis-signaling receptor molecule, related to the family of Tumor Necrosis Factors (TNF), is expressed on the surface of a number of cell types, including liver parenchymal, endothelial, and Kupffer cells. The cells of the liver, including the parenchymal and Kupffer cells constitutively express Fas and are highly sensitive to apoptosis induced by anti-Fas antibody [11]. Fas-mediated apoptosis has been implicated as a contributing factor in liver damage. It has been established that an Fas ligand is elevated in the sera of patients with liver diseases, including chronic hepatitis B and hepatitis C, autoimmune hepatitis and cirrhosis, and in patients with hepatocellular damage resulting from liver transplantation or poisoning [12, 13].

An objective of the present invention is to provide an adequate method for protection of the liver from functional impairment caused by various agents, using an injectable formulation of Idebenone. Anti-Fas antibodies administered in doses of 200 mcg/kg, in mice, causes severe liver inflammation and is immediately reflected by an increase in the liver cell damage marker Alanyl aminotransferase (ALT) from normal levels of 30-50 U/L to >20,000 U/L, as early as 6 hours post-injection. The (invention proposed) developed stable parenteral Idebenone formulation provides noticeable hepatic protection from cellular damage associated with apoptosis which is initiated by anti-Fas antibodies which normally induce acute liver damage.

Such formulation is prepared using an oil-in water emulsion, constituting a mixture of distinct oily components. Idebenone concentrations in the emulsion formulations vary from 0.1% to 2.5% by weight. The oil composition of the emulsion is compounded in a manner such that all incorporated Idebenone is completely dissolved in the discontinuous (oil) phase of the emulsion, avoiding drug precipitation during storage and providing a stable formulation. Compositions with the addition of organic solvents allow for much higher concentrations of Idebenone.

Formulations administrated in intravenous, intraperitoneal or subcutaneous injections during in vivo tests, or added after required dilution to cell culture media during in vitro of ex vivo experiments demonstrate excellent biocompatibility, absence of irritation or toxicity signs and pronounced brain tissue protection.

The following examples are intended to illustrate certain preferred embodiments of the invention and no limitation upon the invention is implied by their inclusion.

Idebenone Formulations

### EXAMPLE 1-10

### Idebenone in Oil-in-Water Emulsions

### Example 1

### Preparation of Injectable Idebenone o/w Emulsion

Oil components of the formulation (Capric/caprylic triglycerides, acetylated monoglycerides and D-alpha-Tocopherol USP) were combined with lecithin and ethloxylated castor oil and mixed at 40° C. for 1 hour. Idebenone was dissolved in warm mixture of oils and surfactants and then blended with water phase, comprising water, EDTA and Glycerin using high shear rotor-stator mixer (5-10,000 rpm, 2 minutes). The obtained emulsion was treated with a high pressure homogenizer (e.g., Avestin™ Emulsiflex C5) at 5,000-15,000 psi (300-1000 bar) for 3-5 cycles. After cooling to room temperature, the emulsion was filtered through a sterile microporous membrane filter (0.2 mcm or 0.45 mcm) in aseptic conditions and dispensed into sterile glass vials. The seated vials were stored in a refrigerator or at room temperature, protected from light. The Idebenone content was tested using HPLC method.

Examples 2-10 of Idebenone loaded o/w emulsions, were prepared in a similar manner, excluding example 8, where the mixture of the oil and water phase as passed through 0.22 mcm microporous membrane 3 times, instead of high pressure homogenization. Compositions of examples 1 through 10 are presented in table 1.

TABLE 1
Idebenone in oil-in-water emulsions (Examples 1-10)

| | 1 | 2 | 3 | 4 | 5 | 6 | 7 | 8 | 9 | 10 |
|---|---|---|---|---|---|---|---|---|---|---|
| | Percentage of composition | | | | | | | | | |
| Idebenone | 1.0 | 2.0 | 1.0 | 0.25 | 0.10 | 0.35 | 2.0 | 0.5 | 0.5 | 2.0 |
| Soya oil | | | 5.0 | 2.0 | | | | | | |
| Capric/caprylic | 12.5 | 18.0 | 10.0 | 8.0 | 10.0 | | 12.0 | | 16.0 | 18.0 |
| triglycerides (MCT) | | | | | | | | | | |
| Tocopherol USP | 0.1 | 8.0 | | | | | 3.0 | 2.5 | | 2.0 |
| Acetylated monoglycerides | 12.5 | | 10.0 | | | 10.0 | 15.0 | | 9.0 | 10.0 |
| Ethyl oleate | | | | | | | | 5.0 | | |
| Polysorbate-80 | | 1.0 | | | | | 0.5 | | 0.1 | 0.5 |
| TPGS | | | 0.5 | | | | | 1.0 | | |
| Ethoxylated castor oil | 0.5 | | | | 0.25 | | | | | |
| Lecithin | 2.0 | 1.5 | 1.8 | 1.0 | 1.0 | 2.0 | 1.5 | 1.0 | 1.5 | 1.5 |
| (phosphatidylcholine > 70%) | | | | | | | | | | |
| Ethanol | | | 1.8 | | | | 1.5 | | | |
| Propylene glycol | | | | | | | | | | |
| Glycerin | 2.5 | 2.25 | | 2.25 | 2.25 | | | | 2.25 | 2.25 |
| Glycine | 0.5 | | | | 0.5 | | | 2.0 | | |
| EDTA | 0.02 | 0.02 | 0.02 | 0.02 | 0.02 | 0.02 | 0.02 | 0.02 | 0.02 | 0.02 |
| PURIFIED WATER | 68.38 | 67.23 | 69.88 | 86.48 | 85.88 | 87.63 | 64.48 | 87.98 | 70.63 | 63.73 |
| to 100% | | | | | | | | | | |
| Total: | 100% | 100% | 100% | 100% | 100% | 100% | 100% | 100% | 100% | 100% |

Examples 11-16 of an Idebenone loaded emulsion, with increased content of oil phase, were prepared by either high pressure homogenization or by spontaneous emulsification in a mixture of the oil, surfactant and stabilizer after addition of water phase, without the homogenization step. For example 11, Idebenone was dissolved with, slight heating (50-55° C.), in an oily mixture of acetylated monoglycerides Myvacet™ 9-45K) and Vitamin E (Tocopherol mix), containing d-alpha tocopheryl polyethylene glycol 1000 succinate (Vitamin F TPGS) surfactant and soy lecithin. Propylene glycol was added to the warm solution and then the water phase, heated to 65-70° C., was added and mixed with the oil composition using a propeller mixer at low speed to avoid foaming. Examples 13 and 15 were prepared in the same manner as example 11, while examples 12, 14 and 16 were treated with a high pressure homogenizer. The formed oil-in-water emulsion was passed through a microporous membrane filter (0.1 mcm) and stored at room temperature. Compositions of examples 11 through 16 are presented in table 2.

TABLE 2
Idebenone in oil-in-water emulsions (Examples 11-16) with
high level of oil phase

| | 11 | 12 | 13 | 14 | 15 | 16 |
|---|---|---|---|---|---|---|
| | Percentage of composition | | | | | |
| Idebenone | 1 | 2 | 2.5 | 2.5 | 2.5 | 2.0 |
| Soybean oil (LCT) | | 28 | 2 | 2 | 2 | 12 |
| Capric/caprylic triglycerides | | | 14 | 8 | | 16 |
| (MCT) | | | | | | |
| Tocopherol USP | 8 | | 8 | 4 | | |
| Acetylated monoglycerides | 14 | | 8 | 16 | 16 | |
| Triacetin | | | | | 10 | |
| Caprylic/Capric mono/di- | | | | | | |
| glycerides | | | | | | |
| Oleic acid | | 0.05 | | | | |
| Polysorbate-80 | | 5 | | 4 | | 0.5 |
| Solutol ® HS-15 | | | | | 4 | |
| TPGS | 5 | | | | | |
| Ethoxylated castor oil | | | 5 | | | |
| (Incrocas-35) | | | | | | |
| Lecithin USP | 1.2 | 2 | 2.5 | 2 | 2 | 2.2 |
| (phosphatidylcholine > 70%) | | | | | | |
| Ethanol | | 2 | 2.5 | | | |
| Propylene glycol | 5 | | 5 | | | |
| Glycerin | | | | 2.25 | 2.25 | |
| Benzyl alcohol | | | | 0.5 | | 2.2 |
| Dibasic sodium phosphate | | | | | 0.4 | 0.4 |
| EDTA disodium | 0.02 | 0.02 | 0.02 | 0.02 | 0.02 | 0.02 |
| Methyl paraben | 0.2 | 0.2 | 0.2 | | 0.2 | 0.2 |
| PURIFIED WATER | 65.58 | 60.73 | 40.28 | 58.73 | 60.63 | 58.98 |
| to 100% | | | | | | |
| Total: | 100% | 100% | 100% | 100% | 100% | 100% |

Formulations 1-16 are stable at room temperature for several months with no signs of phase separation or Idebenone precipitation. The obtained oil-in-water emulsions were passed through microporous membrane filter (0.1 mcm) without loss of Idebenone content.

Examples 17-22 demonstrate potential of preparing highly loaded formulations which can not be used for intravenous administration due to the formation of large particles or droplets after contact with a water media, or due to the highly irritative or hemolytic properties of the solvent composition, but are suitable for intramuscular or subcutaneous administration.

Examples 17-22 are prepared by combining of all components, and slight heating to 40-50° C., until a clear solution is obtained. The prepared solutions are sterilized by filtration through a 0.1 mcm membrane filter. Preparations remain stable at room temperature for several months. After mixing with water a coarse emulsion is formed, and Idebenone precipitates after several hours at room temperature.

TABLE 3
Idebenone in solvent based formulations (Examples 17-22)

| | 17 | 18 | 19 | 20 | 21 | 22 |
|---|---|---|---|---|---|---|
| | Percentage of composition | | | | | |
| Idebenone | 2.5 | 5.0 | 5.0 | 10.0 | 20.0 | 30.0 |
| Ethyl lactate | 20 | | | | | |
| Capric/caprylic | 10 | | | | | 15 |
| triglycerides (MCT) | | | | | | |
| Tocopherol USP | | | | 10 | | |
| Triacetin | | | | 60 | | |
| N-Methylpyrrolidone | | | | | | 50 |
| (Pharmasolve ™) | | | | | | |
| Pyrrolidone-2 | 30 | | | | | |
| DMSO | | 60 | | | | |
| Acetylated monoglycerides | | | 75 | | | |
| Ethanol | | 10 | | | 20 | |
| Propylene glycol | | | 18 | 18 | 25 | |
| Polysorbate-80 | | | 2 | 2 | 5 | 5 |
| Solutol ® HS-15 | | | | | | |
| PEG400 | 35 | 15 | | | 30 | |
| Ethoxylated castor oil | 2.5 | 5 | | | | |
| (Cremophor EL) | | | | | | |
| Benzyl alcohol | | 5 | | | | |
| Total: | 100 | 100 | 100 | 100 | 100 | 100 |

Animal Experiments

Animals (BALB/C mice, 20-22 g) were injected IV with Fas antibodies supplied by BD Pharmingen™ (Purified NA/LE Hamster anti-Mouse CD-95) in a dose 200 mcg/kg. Six hours after antibody administration, blood was collected and the serum analyzed for ALT levels using a Thermo Scientific ALT reagent kit TR 18515, using UV kinetic determinations. The experimental animals were divided into three groups: a control group injected with placebo vehicle, a first experimental group injected with injectable Idebenone (Example 16) in dose 30 mg/kg 15 minutes before antibodies administration, and a second experimental group treated with injectable Idebenone in same dose 30 mg/kg 15 minutes after anti-Fas antibodies delivery.

Increased Alanyl aminotrasferase (ALT) levels reflect damage and death of hepatocytes caused by apoptosis, after administration of antibodies to mouse CD95 protein (anti-Fas antibodies). Six hours after intravenous injection of Fas antibodies in a dose of 200 mcg/kg, ALT levels increase 700 times, from 29 U/L to 22,000 U/L, showing severe liver injury.

The preventive administration of a Idebenone nanoemulsion in a dose of 30 mg/kg intraperitoneally (IP) 15 minutes before Fas antibody injection, demonstrates significant decrease in ALT (˜47%) when compared to a placebo controlled group (p<0.05). The administration of Idebenone in the same dose 15 minutes after Fas antibodies injection demonstrates even more impressive activity: ALT levels decrease by 90% (FIG. 1.) when compared to vehicle only treated animals (p<0.01).

### BRIEF DESCRIPTION OF THE DRAWING

FIG. 1 shows influence of Idebenone on the level of Alanyl aminotransfrase (ALT) in acute anti-Fas induced hepatitis model (mice).

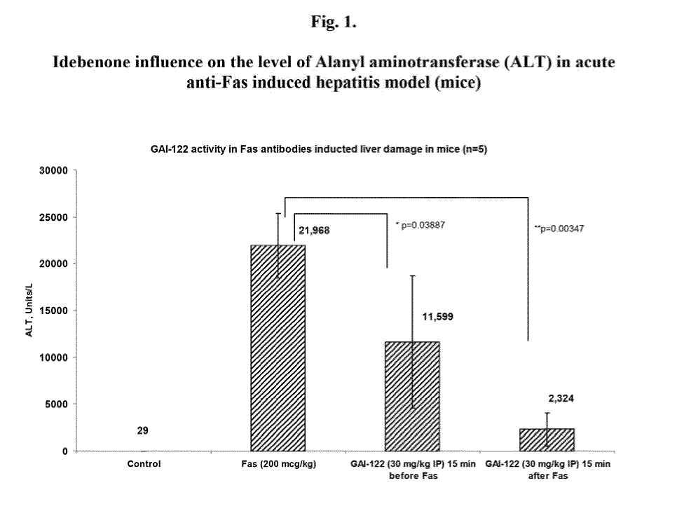

1. A method of prevention or treatment of liver disorders and ameliorating diseases and conditions associated with liver damage; said method comprises the parenteral administration of pharmaceutical compositions, comprised of at least one physiologically acceptable derivative of 1,4-benzoquinone.

2. A method as set forth in claim 1 wherein said composition is a derivative of 1,4-benzoquinone is 2,3-dimethoxy-5-methyl-6-(10-hydroxydecyl)-1,4-benzoquinone (Idebenone).

3. A method as set forth in claim 1 wherein said composition is administrated parenterally via intravenous injection, intravenous infusion, intra-arterial, intramuscular, subcutaneous or intraperitoneal injection.

4. A method as set forth in claim 1 wherein said derivative of 1,4-benzoquinone is administrated in doses ranging from 0.5 to 50 mg/kg per day.

5. A method as set forth in claim 1 wherein said composition is incorporated in a colloidal delivery system, whose components are selected from micellar preparations, emulsions, liposomes, solid lipid nanoparticles, polymeric nanoparticles, nanocapsules or suspensions, wherein said 1,4-benzoquinone derivative is associated with a hydrophobic phase of the colloidal system.

6. A method as set forth in claim 5 wherein said emulsion is an oil-in-water emulsion.

7. A method as set forth in claim 1 wherein said liver disorder is caused by trauma.

8. A method as set forth in claim 1 wherein said liver disorder is caused by poisoning.

9. A method as set forth in claim 1 wherein said liver disorder is caused by apoptosis.

10. A method as set forth in claim 1 wherein said liver disorder is associated with mitochondrial dysfunction.

11. A method as set forth in claim 1 wherein said liver disorder is caused by non-steroidal analgesics or antipyrexics.

12. A method as set forth in claim 1 wherein said liver disorder is caused by ethanol.

13. A method as set forth in claim 1 wherein said liver disorder is caused by antibiotics.

14. A method as set forth in claim 1 wherein said liver disorder is caused by drug abuse.

15. A method as set forth in claim 1 wherein said liver disorder is caused by halogenated hydrocarbons.

16. A method as set forth in claim 1 wherein said liver disorder is associated with liver damage caused by protozoal, fungal, bacterial or viral infections.

17. A method as set forth in claim 16 wherein said liver disorder is caused by hepatitis virus of type A, B, C, D, E, G.

18. A method as set forth in claim 1 wherein said liver disorder is caused by acute hepatitis or acute liver failure.

19. A method as set forth in claim 1 wherein said liver disorder is caused by an inhalational anesthetic agent.

20. A method as set forth in claim 11 wherein said liver disorder is caused by paracetamol/acetaminophen compounds.

21. A method as set forth in claim 11 wherein said liver disorder is caused by salicylate, acetylsalicylate or metamizole.

22. A method as set forth in claim 13 wherein said liver disorder is caused by isoniazid.

23. A method as set forth in claim 13 wherein said liver disorder is caused by tetracyclines.

24. A method as set forth in claim 19 wherein said liver disorder is caused by isoflurane, desflurane, enflurane or sevoflurane.

25. A method as set forth in claim 1 wherein said liver disorder is caused by liver reperfusion and liver transplantation.

26. A method as set forth in claim 1 wherein said liver disorder is caused by acute alcoholic intoxication.

27. A method as set forth in claim 1 wherein said liver disorder is caused by steroid hormones.

28. A method as set forth in claim 1 wherein said liver disorder is caused by aflatoxins or aflastatins.

29. A method as set forth in claim 1 wherein said liver disorder is caused by cholestasis or cholecystitis.

30. A method as set forth in claim 1 wherein said liver disorder associated with an increase in alanyl aminotransferase and aspartate aminotransferase levels.
